(19) United States
(12) Patent Application Publication
(10) Pub. No.: US 2013/0246115 A1
(43) Pub. Date: Sep. 19, 2013

(54) Automatically Starting a Medical Workflow

(71) Applicant: Wolfgang Stempfer, Erlangen (DE)

(72) Inventor: Wolfgang Stempfer, Erlangen (DE)

(21) Appl. No.: 13/800,104

(22) Filed: Mar. 13, 2013

(30) Foreign Application Priority Data
Mar. 14, 2012 (DE) DE 102012203975.9

(51) Int. Cl. G06Q 10/06; G06Q 50/22

(52) U.S. Cl. CPC G06Q 10/063118; G06Q 50/22
USPC 705/7.17

(57) ABSTRACT

The present embodiments relate to starting a role-specific workflow in a medical-technical system. A user of the medical-technical system may be authenticated. A role may be assigned to the user. A replacement of a component of the medical-technical system may be identified using an electronic serial number stored in the component. The role-specific workflow may be started when the replacement of the component is identified. A service delivery may be more heavily standardized and optimized by automatically assigning the workflow. As a result, service results may improve and customer satisfaction may increase.

This application claims the priority benefit of DE 10 2012 203 975.9, filed on Mar. 14, 2012, which is hereby incorporated by reference.

### BACKGROUND

The present embodiments relate to starting a role-specific workflow during operation of a medical-technical system.

Medical-technical systems are generally highly modern structures that have a plurality of components and complex software. Examples of medical-technical systems are x-ray devices in the field of medical imaging application. Digital image recording techniques, in which the x-rays penetrating an object positioned in the examination volume are detected in a locally resolved manner in order to generate a corresponding locally-resolved x-ray image, are used in this field. The x-ray computed tomography technique enables almost any slice images through the examination object to be reconstructed and shown to the user.

A computed tomograph includes, inter alia, an x-ray tube, x-ray detectors and a patient support couch. The x-ray tubes and the x-ray detectors are arranged on a gantry, which, during the measurement, rotates about the patient support couch and/or an examination axis (e.g., the z-axis) parallel thereto. The patient support couch may be moved relative to the gantry along the examination axis. The x-ray tube generates an x-ray beam fanned out in a slice layer at right angles to the examination axis. This x-ray beam penetrates a slice of an object, during examinations in the slice layer (e.g., a body slice of a patient on the patient support couch), and strikes the x-ray detectors facing the x-ray tube. The angle at which the x-ray beam penetrates the body slice of the patient and the position of the patient support couch relative to the gantry may change continuously during the image recording using the computed tomograph.

The intensity of the x-rays of the x-ray beam that strike the x-ray detectors after penetrating the patient is dependent on the attenuation of the x-rays through the patient. Each detector element of the x-ray detectors generates a voltage signal as a function of the intensity of the received x-rays. The intensity corresponds to a measurement of the global transparency of the body for x-rays from the x-ray tube to the corresponding x-ray detector element. A set of voltage signals of the x-ray detectors, which correspond to attenuation data and are recorded for a special position of the x-ray source relative to the patient, is referred to as a projection. A set of projections, which are recorded at various positions of the gantry during the rotation of the gantry about the patient, is referred to as a scan. The computed tomograph records a number of projections at various positions of the x-ray beam source relative to the body of the patient and, in turn, reconstructs an image that corresponds to a two-dimensional slice image of the body of the patient or to a three-dimensional image. The current method of reconstructing a slice image from recorded attenuation data is known as filtered back projection.

The prior art discloses combining medical-technical systems, such as the computed tomographs described above, with a remote service center using modern information technology (e.g., remote service networks). Services, for which use on site was previously required, may thus be enabled via data line. A preventative service, instead of a reactive service, of medical-technical systems is thus possible. Weak points are promptly detected before they result in serious errors by pro-actively monitoring the systems. The availability of the medical-technical system increases and downtimes are reduced. A service may take place by accessing the hardware and software “remotely.” Remote service networks also assist a service engineer on site by, for example, providing information.

Medical-technical systems are operated by different users, such as, for instance, medical personnel or service engineers, to which different user roles are assigned. The role of the medical-technical system may be automatically identified.

The role defines tasks, properties and, above all, rights of a user in a software and/or operating system of the medical-technical system. Roles are used in order to avoid separately defining the settings of the software and/or operating system for each user. Instead of directly assigning rights to users, a role is defined which may then be assigned to various users. This facilitates the administration of user privileges of the software system, since, particularly with changes in the rights structure, only the rights of the role have to be adjusted. Modern software systems provide the user with a graphic user interface adjusted to his/her role and an adjusted workflow. For instance, a repair workflow is provided to service technicians. Identifying a role and the associated workflow is essentially function-based.

A workflow combines the persons and/or work processes that are occurring in a work process in a process model. Workflow management intends to structure, operationalize, and optimize internal resources and activities of employees within a business process according to fixed parameters.

### SUMMARY AND DESCRIPTION

The present embodiments may obviate one or more of the drawbacks or limitations in the related art. For example, a method, a medical-technical system, and/or a computer program product that improves the automatic starting of a role-specific workflow may be provided.

The present embodiments may automatically identify a replacement of a component and/or query fault patterns and automatically start a workflow based on the automatic identification of the replacement of the component and/or the queried fault patterns.

In one or more of the present embodiments, a computer-implemented method for starting a role-specific workflow in a medical-technical system after replacing a component of the medical-technical system may be provided. The method includes the steps of: authenticating a user of the medical-technical system; assigning a role to the user; identifying the replacement of the component using an electronic serial number stored in the component; and starting the role-specific workflow when the replacement of a component is identified.

The service delivery is more heavily standardized and optimized by automatically assigning the workflow to a role. As a result, service results will improve and customer satisfaction will increase.

In one embodiment, the workflow may be specifically adjusted or tailored to the replaced components.

In one or more of the present embodiments, a computer-implemented method for assigning a role-specific workflow in a medical-technical system with or using queried fault patterns may be provided. The method includes the steps of authenticating a user of the medical-technical system, assigning a role to the user; querying, via the user, at least one fault pattern, and starting the role-specific workflow as a function of the at least one queried fault pattern.

The service delivery is more heavily standardized and optimized by automatically assigning the role-specific workflow. As a result, service results will improve and customer satisfaction will increase.

In one embodiment, the workflow may be user-specific.

In one or more of the present embodiments, a medical-technical system for starting a role-specific workflow may be provided. The medical-technical system includes an authentication module configured to identify and/or authenticate a user, an assignment module configured to assign a role to the user, at least one identification module configured to identify a replacement of a component of the medical-technical system with or based on an electronic serial number stored in the component, and a start module configured to start the workflow when the replacement of a component is identified. The serial number may be stored in an EEPROM of the component.

In one or more of the present embodiments, a medical-technical system for starting a role-specific workflow may be provided. The medical-technical system includes an authentication module configured to identify and/or authenticate a user, an assignment module configured to assign a role to the user, a querying module configured to query the fault pattern, and a start module configured to start the workflow as a function of the queried fault pattern.

In one embodiment, the medical-technical system may be a computer tomograph or a magnetic resonance tomograph.

In one or more of the present embodiments, a computer program product may be provided. The computer program product has instructions that may be loaded into a memory of a programmable medical-technical system and executed in the medical-technical system to start a role-specific workflow in a medical-technical system with or using queried fault patterns and/or after replacing a component of the medical-technical system.

### BRIEF DESCRIPTION OF THE DRAWINGS

FIG. 1 shows one embodiment of a computer-implemented method for automatically starting a repair workflow in a medical-technical system;

FIG. 2 shows another embodiment of a computer-implemented method for automatically starting a workflow in a medical-technical system based on one or more fault patterns; and

FIG. 3 shows a block diagram of one embodiment of a medical-technical system for automatically assigning a workflow.

### DETAILED DESCRIPTION OF THE DRAWINGS

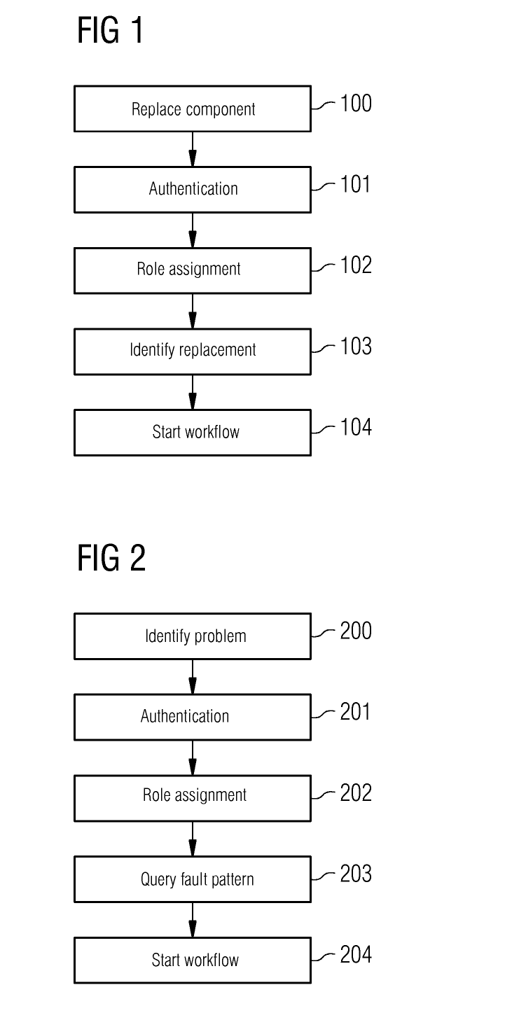

FIG. 1 shows one embodiment of a method in which a repair workflow is automatically assigned to a service technician. With a medical-technical system (e.g., a computed tomograph), a component is replaced by a service technician (act 100). The service technician then logs into the medical-technical system and is automatically authenticated as a service technician (act 101). The service technician is assigned his/her associated role (act 102). This role provides the service technician with special authorizations and workflows.

The medical-technical system identifies the replacement of the component with or based on an electronic serial number or identification number that is stored in the component (act 103). For instance, the serial number is stored in an EEPROM of the component and is read in when a component is replaced. Subsequently, a repair workflow associated with the user role and specific to the component is started (act 104). The service technician is now intentionally controlled by necessary work steps, such as adjustment processes and settings to be carried out, and obtains all necessary information (e.g., a temperature indicator for an adjustment phantom) on or from a monitor.

In one embodiment, communication with a remote service center may also be set up for assistance purposes.

FIG. 2 shows one embodiment of a method in which a workflow is started based on one or more fault patterns. A user of the medical-technical system first identifies a problem (act 200). The user registers with the system and is authenticated (act 201). A user role is then assigned to the user (act 202).

In step 203, the authenticated user queries one or more fault patterns on the medical-technical system or in a remote service center communicatively connected with the system. Based on the one or more queried fault patterns, a workflow is started (act 204). The workflow is specific to the one or more queried fault patterns and to the user role. A user is therefore intentionally assigned a workflow that “guides” him/her through the medical-technical system and thus allows the problem to be limited or eliminated.

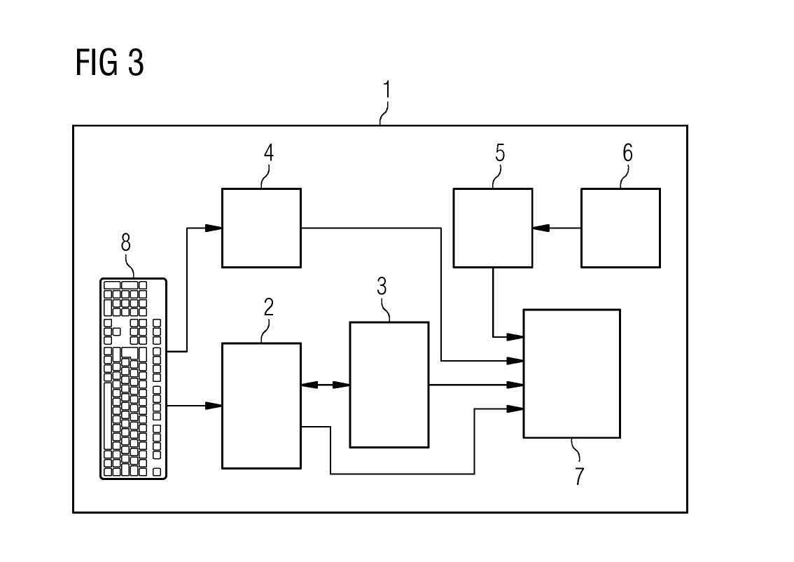

FIG. 3 shows a block diagram of one embodiment of a medical-technical system 1 for automatically starting a workflow. The medical-technical system 1 includes an authentication module 2 configured to identify and authenticate a user of the system. The system also includes an assignment module 3 configured to assign a user role to the user, and a query module 4 configured to query one or more fault patterns. The one or more fault patterns may be queried by the user using, for example, an input unit 8. Furthermore, the system 1 includes at least one identification module 5 configured to identify a replacement of a component 6 of the medical-technical system 1 with or using an electronic serial number stored in the component 6.

The workflow is started with a start module 7 when the replacement of a component 6 is identified or one or more fault patterns are queried. The workflow is specific to the replaced component 6 or specific to the queried fault patterns. The workflow is also role-dependent and may be user-specific.

The electronic serial number may be stored in an EEPROM of component 6. The identification module 5 reads the serial number out from the EEPROM. Modules 2 to 5 and 7 may be or include hardware and/or software components.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A computer-implemented method for starting a role-specific workflow in a medical-technical system after replacing a component of the medical-technical system, the computer-implemented method comprising:
authenticating a user of the medical-technical system;
assigning a role to the user;
identifying a replacement component using an electronic serial number stored in the component; and
starting the role-specific workflow when the replacement component is identified.

2. The computer-implemented method as claimed in claim 1, further comprising tailoring the role-specific workflow to the replaced component.

3. A computer-implemented method for starting a role-specific workflow in a medical-technical system using at least one queried fault pattern, the computer-implemented method comprising:
authenticating a user of the medical-technical system;
assigning a role to the user;
querying the at least one fault pattern; and
starting the role-specific workflow as a function of the querying.

4. The computer-implemented method as claimed in claim 3, wherein querying the at least one fault pattern is performed by the user.

5. The computer-implemented method as claimed in claim 3, wherein starting the role-specific workflow comprises starting the role-specific workflow as a function of the querying and the user.

6. A medical-technical system for starting a role-specific workflow, the medical-technical system comprising:
an authentication module configured to authenticate a user;
an assignment module configured to assign a role to the user;
at least one identification module configured to identify a replacement of a component of the medical-technical system using an electronic serial number stored in the component; and
a start module configured to starts the role-specific workflow when the replacement of the component is identified.

7. The medical-technical system as claimed in claim 6, wherein the start module is configured to specifically tailor the role-specific workflow to the replaced component.

8. A medical-technical system for starting a workflow, the medical-technical system comprising:
an authentication module configured to authenticate a user;
an assignment module configured to assign a role to the user;
a query module configured to query at least one fault pattern; and
a start module configured to start the role-specific workflow as a function of the at least one queried fault pattern.

9. The medical-technical system as claimed in claim 8, wherein the medical-technical system is a computed tomograph or a magnetic resonance tomograph.

10. The medical-technical system as claimed in claim 8, wherein the work-flow is user-specific.

11. A non-transitory computer-program product that includes instructions that can be loaded directly into a memory of a programmable medical-technical system and executed by the programmable medical-technical system to start a role-specific workflow in the medical-technical system after replacing a component of the medical-technical system, the instructions comprising:
authenticating a user of the medical-technical system;
assigning a role to the user;
identifying a replacement component using an electronic serial number stored in the component; and
starting the role-specific workflow when the replacement component is identified.

12. The computer-program product as claimed in claim 11, the instructions further comprising tailoring the role-specific workflow to the replaced component.

13. A non-transitory computer-program product that includes instructions that can be loaded directly into a memory of a programmable medical-technical system and executed by the medical-technical system to start a role-specific workflow in a medical-technical system using at least one queried fault pattern, the instructions comprising:
authenticating a user of the medical-technical system;
assigning a role to the user;
querying the at least one fault pattern; and
starting the role-specific workflow as a function of the querying.

14. The computer-program product as claimed in claim 13, wherein querying the at least one fault pattern is performed by the user.

15. The computer-program product as claimed in claim 13, wherein starting the role-specific workflow comprises starting the role-specific workflow as a function of the querying and the user.
